(19) United States
(12) Patent Application Publication
(10) Pub. No.: US 2022/0304742 A1
(43) Pub. Date: Sep. 29, 2022

(54) MEDICAL INSTRUMENT AND METHOD AND MODULAR SYSTEM FOR MANUFACTURING OF SUCH INSTRUMENTS

(71) Applicant: Erbe Elektromedizin GmbH, Tuebingen (DE)

(72) Inventors: Stefan Kaupp, Tuebingen (DE); Felix Bob, Rottenburg (DE)

(73) Assignee: Erbe Elektromedizin GmbH, Tuebingen (DE)

(21) Appl. No.: 17/698,122

(22) Filed: Mar. 18, 2022

(30) Foreign Application Priority Data
Mar. 25, 2021 (EP) 21164843.1-1113

(51) Int. Cl. A61B 18/14

(52) U.S. Cl. CPC A61B 18/1442; A61B 2018/146; A61B 2018/00589

(57) ABSTRACT

An instrument capable of being repeatedly sterilized. The instrument has handle pieces that are configured as a housing. The housing includes housing shells that are joined to one another at butt joints without a gap. The interior of the butt joints may be completely filled with a casting compound.

### CROSS-REFERENCE TO RELATED APPLICATION

This application claims priority to European Application No. 21164843.1-1113, filed Mar. 25, 2021, the entirety of which is incorporated by reference herein.

### TECHNICAL FIELD

Embodiments of the invention refer to a medical instrument, a modular system for manufacturing such instruments as well as a method for providing instruments according to embodiments of the invention.

### BACKGROUND

Medical instruments used on patients frequently consist of a plurality of components that are more or less non-releasably connected with each other. For example, WO 00/27294 A1 discloses a medical instrument for cutting of tissue in human or animal body that is configured in the type of scissors. The scissors comprise scissors arms consisting of metal and provided with an electrical insulation.

An autoclavable handle is known from DE 10 2018 004 244 A1 comprising a housing in which a switching element and further elements are arranged. The switching element is sealed in the handle housing by means of a surrounding sealing element. In order to allow totally automatic cleaning, the housing of this handle is free of joints, whereby switching elements are provided that can be spring-elastically pressed into the handle housing. The surrounding sealing is formed from an autoclavable material and bridges the gap between the movable switching element and the rigid handle housing in a sealing manner. The hollow spaces formed in the handle are thus largely inaccessible from outside.

An autoclavable remote control having a two-part housing is known from DE 10 2004 041 871 B4 in which a board is arranged that is coated with silicone and casting compound. For manufacturing the remote control first the board provided with components is hermetically cast into the casting compound, whereby mechanical elements of the remote control project slightly from the casting compound. Subsequently, a top part is adhesively bonded to the casting compound, wherein at least one push button is surrounded by defined air entrapment.

A sterilizable dentist handle having an electrical coil is known from DE 694 18 799 T2 that is configured to be sterilized by an arbitrary current sterilization method in the assembled condition. The handle is thereby configured such that a housing element or shell as well as a coil unit loosely fit together in the assembled condition at the front end of the coil unit so that sterilization fluids, such as vapor, chemicals or heat, can enter in the interior for quicker and more exhaustive sterilization of the entire handle. For this one or more openings or gaps shall be provided on the handle that extend into the interior of the handle.

An electrosurgical instrument for cutting of tissue is known from EP 1 769 764 A2 as well as U.S. Pat. No. 9,498,279 A2 respectively comprising two scissors arms. The scissors arms are configured as housings surrounding hollow spaces with metal parts arranged therein respectively. The housings are assembled from housing shells that can be adhesively bonded with each other.

EP 2 630 982 A1 describes a fluid connector via which an instrument is to be supplied with a fluid. The connector is configured as housing through which individual lines extend. The interior of the connector is filled with casting compound.

### SUMMARY

Numerous requirements are posed on electrosurgical instruments. Apart from a reliable often repeated sterilization ability, geometrical precision of the instrument and the functional units provided thereon and the mechanical strength are also frequently important. For example, electrodes provided on the instrument have to be precisely positioned with regard to one another or with regard to other elements, such as hinges. Also frequently important is a uniform insulation that is sufficiently thick everywhere.

Starting therefrom it is the object of the embodiments of the invention to provide an improved electrosurgical instrument, a modular system for providing such instruments as well as a method for manufacturing the same.

This object is solved, for example, by means of an instrument according to claim 1, the modular system according to claim 12 as well as the method according to claim 14:

The instrument according to embodiments of the invention comprises at least one handle piece that consists of a housing having multiple housing shells. The housing shells surround an interior in which (at least one) inner part is arranged, e.g. a metal inlay. Between the inner part and the housing an interstice is formed that is filled with a casting compound free of air entrapments.

With this concept a simple and concurrently precise production of electrosurgical instruments can be provided. Inner parts, e.g. stabilizing metal inlays, inlays made of fiber compound material, particularly fiber-reinforced plastic or the like, can be inserted into the housing shells and then overmolded, whereby preferably easily flowing and gap-filling casting compound is used. The casting compound can thus be introduced in a pressureless manner without the danger of dislocation of used inner parts. Different to inserting of metal parts, cables or other functional elements in an injection molding die and overmolding the parts with ductile plastic the danger of waste part production resulting from displacement or pushing aside of inner parts (metal inlays, cables, switches and the like) due to the plastic injected with high pressure (up to 300 bar) during injection molding does neither exist in the instrument according to embodiments of the invention nor in the method according to embodiments of the invention. The used inner parts can be held at locations as necessary that are later accessibly positioned and held as function parts. In this manner jaws of scissors or coagulation forceps can be manufactured, for example, in which a longitudinal metal part extends from a distal end via a hinge area up to a proximal end. The metal part is first inserted in the housing assembled from multiple housing shells and is positioned, e.g. on the hinge or on the accessible areas that later serve as electrodes, after which the casting compound is filled in the housing and cured therein.

Due to the complete filling of the interior with casting compound entering of germs, dirt or other contaminations in the interior of the housing is avoided. Gaps or hollow spaces are completely filled. This facilitates sterilization remarkably. Introduction of the still liquid casting compound into the interior can be carried out in that bowl-shaped housing parts are first filled with liquid casting compound, then the inlays are inserted and subsequently the housing parts are joined together. Preferably the casting compound is, however, only filled into the interior of the instrument after assembly of the instrument. Filling openings can be provided in one or multiple of the housing shells for this purpose via which the casting compound is inserted into the interior. Preferably the casting compound is a compound that does not shrink during curing that connects the housing shells and the inner parts by adhesive bond, e.g. a casting resin.

Preferably the housing shells consist of a plastic that is stiffer than the casting compound. The casting compound can compensate different thermal expansion coefficients between the housing shells and the inner parts consisting of metal due to its elasticity. No cracks or gaps form between the housing shells and the inner part due to thermal stresses during autoclaving in which germs could enter. Thus, the instrument can be reliably sterilized many times.

Preferably the housing shells are connected with one another at their edges, where they are in contact with one another by substance bond without gaps. Preferably it is a non-releasable connection, i.e. a connection that cannot be released non-destructively. For this purpose the housing shells can be adhesively connected with one another by means of adhesive, whereby the adhesive joints are sealed to the exterior. Also the casting compound can be used to adhesively connect the housing shells with one another. As an alternative or in addition, the housing shells can be welded to one another at their butt joints, e.g. by laser welding, ultrasonic welding, friction welding or the like.

The concept according to embodiments of the invention allows the configuration of the housing shells with uniform wall thickness respectively, e.g. by means of an injection molding method. The housing shells can thus be manufactured with high precision. The different distances between the inner part and the outer surface of the housing shells are bridged by a casting compound. It is also possible to manufacture the housing shells by means of an additive manufacturing technique. Particularly instruments having the same inner parts, but different housings can be provided as needed thereby (e.g. instruments for hands of different sizes or for right- and left-handers).

The concept according to the embodiments of the invention provides remarkably stiff handle pieces, because the housing shells support to the total stiffness of the handle piece due to their inherent stability. Thus, the housing shells can be used for transmission of actuating forces. Preferably after curing the casting compound forms a force transmitting connection between the housing and the inner part that preferably consists of metal.

The inner part can be entirely electrically insulated toward the exterior and can be without electrical function. It is also possible to form an electrode provided on the instrument as part of the inner part. The configuration concept according to embodiments of the invention allows both.

The instrument according to embodiments of the invention can comprise two handle pieces configured according to the principle explained above that are hingedly supported on one another. For example, they can be the jaws of a forceps or scissors.

The concept according to embodiments of the invention allows in addition the configuration of a modular system with which different instruments can be composed from a variety of different housing shells of a respective stock. For example, these different instruments distinguish from one another only with regard to one or a few housing features. Because they are manufactured without the requirement to provide a separate injection molding die for the entire housing of the instrument respectively, the manufacturing effort is reduced. For example, the housing shell that comprises the varying housing feature can be made by means of additive manufacturing. Then the housing is a combination of at least one additively manufactured housing shell and at least one housing shell manufactured by injection molding. Additive manufacturing methods are particularly methods operating without casting die, such as 3D-printing, selective laser hardening, selective laser sintering or the like. It is, however, also possible to manufacture all of the housing shells by injection molding or all of the housing shells in additive manufacturing manner. Even if the housing shells having the varying housing feature in the series are not additively manufactured without casting die, but by means of an injection molding die, an efficiency advantage results. For example, coagulation forceps with different hand piece sizes can be provided in any of the indicated manners.

### BRIEF DESCRIPTION OF THE DRAWINGS

Further details of embodiments of the invention are derived from the claims as well as the drawings and the associated description. The figures of the drawings show:

FIG. 1 an electrosurgical instrument in schematic perspective illustration,

FIG. 2 the instrument of FIG. 1 in a perspective explosion illustration in part,

FIG. 3 a jaw of the instrument according to FIG. 1 in cross-section,

FIG. 4 an enlarged illustration of the cross-section of the instrument according to FIG. 3 in part,

FIG. 5 a modified embodiment of a butt joint between two housing shells of the instrument according to FIG. 1,

FIG. 6 an electrode having a cable connected thereto for an instrument according to FIG. 1,

FIG. 7 a cross-sectional illustration of the distal end section of a modified embodiment of an instrument according to FIG. 1, and

FIGS. 8 to 10 handle shells for a handle area of a modular system for providing different instruments of the same type, but with different hand piece sizes in schematic perspective illustration respectively.

### DETAILED DESCRIPTION

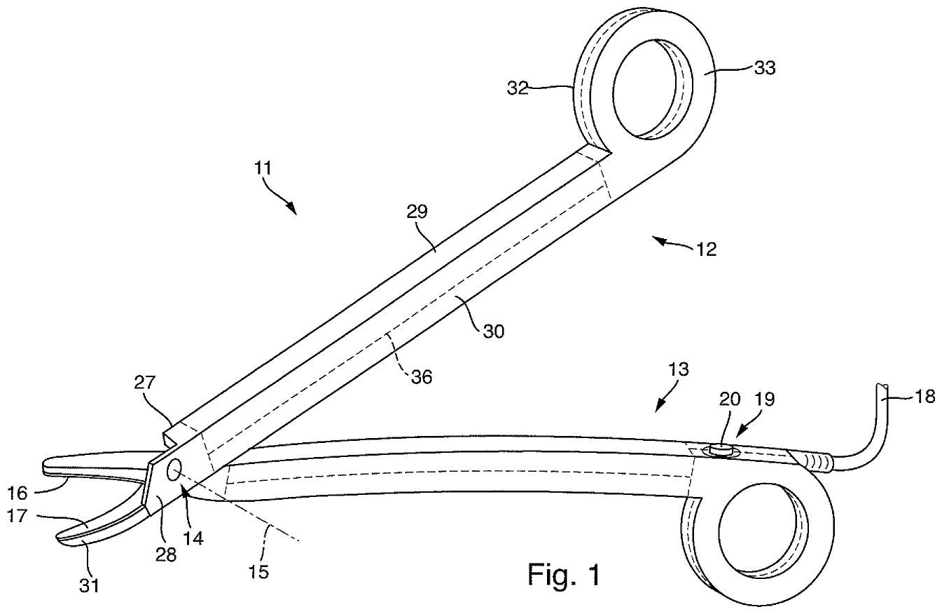

In FIG. 1, an instrument 11 is illustrated that is configured here by way of example as cauterization forceps for the open surgical use. According to the configuration and construction principle of instrument 11 explained in the following, however, also other instruments can be configured, particularly for the open surgical use, such as tissue scissors or combined coagulation and cutting instruments. Particularly, these are instruments for the electrosurgical use during which current is introduced into the tissue by means of the instrument in order to achieve a surgical effect. The instruments 11 configured according to embodiments of the invention are preferably configured as reusable sterilizable instruments.

The instrument 11 comprises at least one or as shown in FIG. 1, two (or more) handle pieces 12, 13 that are configured in the instrument 11 according to FIG. 1 as two jaws that are supported on one another in a hinged manner. The handle pieces 12, 13 are supported on one another in a hinged manner on a hinge 14. The hinge 14 defines a (one single) hinge axis 15 that is orientated transverse to the handle pieces 12, 13.

The instrument 11 comprises at least one, preferably multiple, e.g. two, electrodes 16, 17 that are arranged on the respective part of this handle piece 12, 13 extending away from the hinge 14 in distal direction. The electrodes 16, 17 are insulated with regard to one another and are, as required, supplied with current via a cable 18 that establishes a connection between the electrodes 16, 17 and a not illustrated electrosurgical generator. The cable 18 can originate from a proximal end of one of the two handle pieces 12, 13. In other embodiments a respective insulated line (cable) can be arranged on each handle piece 12, 13.

The instrument 11 can comprise additional electrical or electronic components. In the simplest case an electrical switch 19 is provided on one of the handle pieces 12, 13, for example, preferably on the handle piece 13 on which the cable 18 is arranged, wherein switch 19 is configured to allow or block the current flow toward electrodes 16, 17. This optionally provided switch 19 can be configured in the type of a push-button, for example, that comprises a movable plunger 20. Plunger 20 can face the other handle piece 12 and can be arranged such that it is actuated as soon as handle piece 12 abuts on plunger 20 and is pushed down.

The two handle pieces 12, 13 are configured according to the principle explained based on handle piece 12. Handle piece 12 consists of a multi-part housing 21 surrounding an inner part 22. For example, the inner part is configured of metal and can extend from the distal end of instrument 11 up to the proximal end thereof. For example, inner part 22 is configured as two arm lever that comprises an opening 23 in the area of hinge 14. The distal end of this inner part 22 can comprise an electrode section 24 that is separated from a support section 26 by means of a groove 25 extending on both flanks.

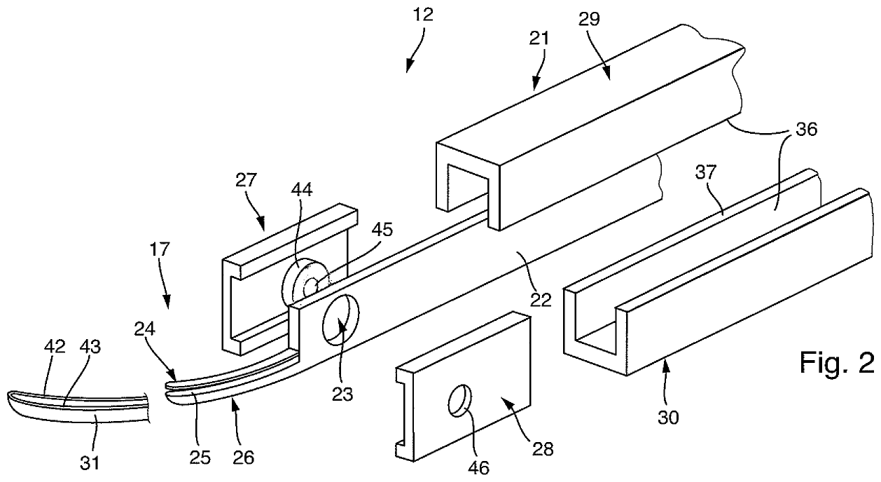

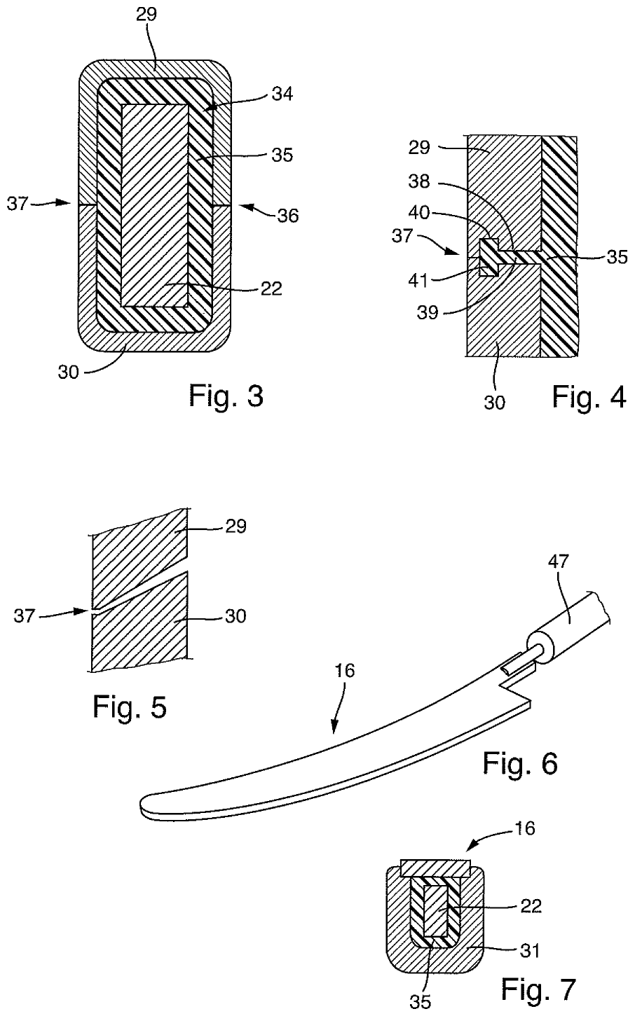

The housing 21 consists of multiple housing shells 27 to 31 as well as 32, 33 (FIGS. 1 and 2) that are joined together without gaps to surround an interior 34 in which the inner part 22 is arranged, as illustrated in FIG. 3.

The housing shells 27 to 33 are preferably made of plastic, e.g. a stiff plastic that is smooth on the outside and that is temperature-resistant for the usual sterilization temperatures of, for example, 130° C. or up to 170° C. The housing shells 27 to 33 can be manufactured by injection molding and can comprise largely uniform wall thicknesses. Alternatively some or all of the housing shells 27 to 33 can be manufactured by an additive manufacturing process without use of a casting die defining the shell shape. As plastic for the housing shells 27 to 33 polyether ether ketone (PEEK), polyaryletherketone (PAEK), semi-crystalline thermoplastic construction materials, e.g. based on polyphtalamide (PPA), polyarylamide (PARA) with or without glass fiber reinforcement, photopolymers on methacrylate basis or other plastics are suitable that are at least thermally resistant up to 140° C.

The interior 34 limited by housing shells 27 to 33 is preferably filled with a casting compound 35. This casting compound 35 fills interior 34, preferably completely and without gaps. This also applies, if instead of one single inner part 22, multiple inner parts are provided that are arranged next to one another or are arranged one after the other. Any temperature-resistant curable plastic is suitable as casting compound 35 that establishes a substance bond connection with the plastic material of housing shells 27 to 33. Particularly, casting compound 35 can consist of the same plastic as housing shells 27 to 33, particularly if it is a cold curable plastic. The casting compound 35 can also be a curable epoxy resin adhesive or epoxy resin casting compound, a cyan adhesive or in general any adhesive having a temperature resistance of at least 140° C.

The housing 21 is preferably completely closed toward the exterior. The housing shells 27 to 33 thereby abut at butt joints 36, 37 against one another illustrated in dashed lines in FIG. 1 and apart therefrom apparent from FIGS. 3 to 5 and are preferably unreleasably connected with one another at the butt joints 36, 37. For example, the used casting compound 35 is suitable to enter into the butt joints in liquid non-cured condition, as by way of example illustrated in FIG. 4 based on butt joint 37. For better and tighter connection of housing shells 27 to 33 with one another, the faces 38, 39 of housing shells 29, 30 (as well as of the other housing shells) can be profiled. For example, the faces 38, 39 can be provided with longitudinal grooves 40, 41 that fill with casting compound 35 during insertion of casting compound 35. After curing of casting compound 35 it adhesively connects the housing shells overall and at the butt joint 37 with each other. Inside grooves 40, 41 it forms a barrier against entering of liquids and germs in possible gaps that may still be present in the interior of instrument 11.

The configuration of butt joint 37 (as well as the further butt joints) can vary and depend on the used connection technique of housing shells. For example, butt joint 37 can be wedge-shaped, as illustrated in FIG. 5 and can be arranged inclined with respect to the horizontal joint, in order to allow a simple joint on one hand and a simple filling of the butt joint 37 with casting compound on the other hand.

In the embodiment described so far, housing shells 27 to 33 are adhesively connected to one another by means of casting compound 35. It is, however, also possible to separately adhesively connect butt joints 36, 37 (and all others) by means of an adhesive first and to only subsequently fill casting compound into the interior 34. It is further possible to close the butt joints 36, 37 by other means and thus to connect the housing shells 27 to 33 with one another, e.g. by ultrasonic welding, laser welding, friction welding or the like.

Housing shells 27 to 33 are configured differently. For example, housing shell 31 (FIG. 2) can be configured to hold support section 26, however, thereby leave electrode section 24 uncovered. Housing shell 31 can thus be configured as shoe that is to be shifted over support section 26. The upper edges 42, 43 of housing shell 31 can thereby comprise a strip projecting inwardly that fits into groove 25. Additional housing shells 27, 28 can envelope and form the hinge area of instrument 11. For example, housing shell 27 can comprise a flat, U-shaped cross-section as well as an approximately centrally arranged extension 44 that fits into opening 23 with or without play. The extension 44 can define a hinge opening 45 provided with an opening 46 aligned with hinge opening 45.

The housing shells 27, 28 define butt joints arranged on top of and below inner part 22. Housing shells 29, 30 are provided that proximally adjoin housing shells 27, 28 the butt joints 36, 37 of which are horizontal. Housing shells 32, 33 are arranged at the proximal end that surround openings and thus are configured as finger grips.

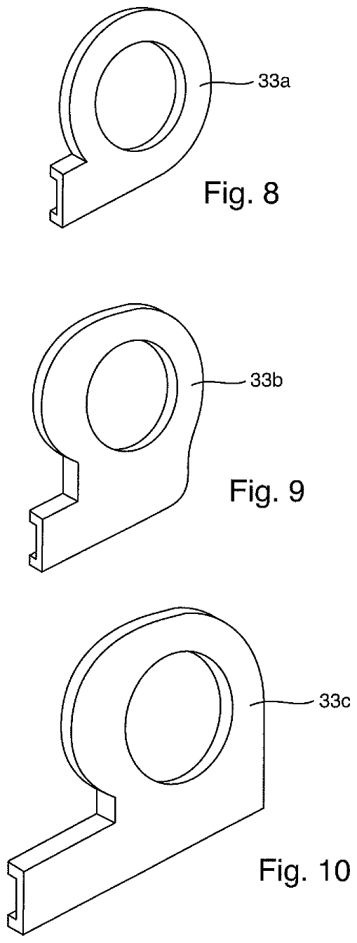

The housing shells 27 to 33 can form a housing shell set that comprises for one and the same position of housing 22 housing shells that are configured differently. This is illustrated by way of example for housing shell 33 in FIGS. 8-10. Different housing shells 33a, 33b, 33c are illustrated there that are likewise suitable to be arranged on the proximal end of housing 21 and thus instrument 11. The housing shells 33a, 33b, 33c distinguish in detail, e.g. in size and/or shape. They are however equally suitable to adjoin housing shells 28, 30. In this manner a housing shell construction set is provided with which instruments 11 with different end shapes or for different finger sizes can be manufactured in a simple manner. Also different lengths of the handle pieces 12, 13 or differences with regard to other housing features can be realized in this manner.

In order to realize housings shaped differently, alternative forms can also be provided for the other housing shells 27 to 31.

Modifications of the provided instruments 11 are possible. While electrode 17 (and 16) are realized by inner part 22, namely its electrode section 24, in the instrument 11 according to FIG. 2, electrode 16 (or 17) can also be configured as separate metal part electrically insulated from inner part 22 as obvious from FIGS. 6 and 7. An insulated line 47 can be connected thereto that extends along the inner part 22 through gripping piece 13 up to switch 19 and/or cable 13.

The instrument 11 is provided as follows.

First, inner part 22 and housing shells 27 to 33 are joined together. Provided that for the housing shells 27 to 33 alternative housing shells are provided, as for example housing shells 33a to 33c according to FIGS. 8-10, the desired housing shells are selected from this stock. After joining of housing shells the created interior 34 is completely filled with casting compound 35 that is subsequently cured inside the interior 34. After curing of casting compound 35 instrument 11 is completed and ready to use.

The housing shells 27 to 31 can be made of the same plastic or also of different plastics. Particularly a different plastic can be used for housing shell 31 than for the other housing shells, for example. The same applies for housing shells 27, 28. Housing shells 27 to 33 can, however, also be made of identical plastic. Preferably they consist of temperature-resistant smooth plastic. The casting compound 35 is preferably thin in non-cured condition and slightly elastic after curing. Preferably it has a larger elasticity than the housing shells 27 to 33. It connects inner part 22 and housing shells 27 to 33 permanently and without gaps.

Due to the elasticity of casting compound 35 as well as the temperature resistance thereof and of housing 21, the instrument 11 can be repeatedly thermally sterilized without having to fear degradation.

The instrument 11 that can be sterilized many times comprises handle pieces 12, 13 that are configured as housing 21. The housing 21 consists of housing shells 27 to 33 that are joined to one another at butt joints without gap and the interior thereof is completely filled with casting compound 35. The instrument is little prone to contamination and easily sterilizable.

1. A sterilizable electrosurgical instrument comprising:
at least one handle piece that comprises a housing that is assembled from multiple housing shells and that surrounds an inner part wherein an interstice is arranged between the inner part and the housing, the interstice being filled with a casting compound.

2. The instrument according to claim 1, wherein the interior is completely filled with the casting compound free of air entrapments.

3. The instrument according to claim 1, wherein at least one electrical component is arranged in the interstice.

4. The instrument according to claim 1, wherein the housing shells comprise a plastic that is stiffer than the casting compound.

5. The instrument according to claim 1, wherein the housing shells are connected with one another in a substance bond manner.

6. The instrument according to claim 1, wherein the housing shells have a uniform wall thickness respectively.

7. The instrument according to claim 1, wherein the inner part comprises a metal.

8. The instrument according to claim 7, further comprising:
at least one electrode.

9. The instrument according to claim 8, wherein the at least one electrode is insulated from the inner part.

10. The instrument according to claim 8, wherein the at least one electrode and the inner part are electrically connected with each other and are realized as one part.

11. The instrument according to claim 1, further comprising:
two handle pieces that are supported on one another in a hinged manner.

12. A modular system for providing medical instruments according to claim 1, the modular system comprising a stock of different configured housing shells usable at the same location on the housing.

13. The modular system according to claim 12, wherein the differently configured housing shells are handle shells.

14. A method for manufacturing medical instruments, the method comprising:
arranging at least one inner part in an interior;
joining the at least one inner part and at least two housing shells such that the at least two housing shells surround the interior;
filling the interior surrounded by the at least two housing shells with a casting compound; and
curing the casting compound.

15. The method according to claim 14, further comprising:
forming joints between the housing shells; and
completely filling the joints with the casting compound.
